(19) United States
(12) Patent Application Publication
(10) Pub. No.: US 2004/0244087 A1
(43) Pub. Date: Dec. 9, 2004

(54) Body-supported air deflector for motorcycle riders

(76) Inventor: Jack G. Halterman, Minden, LA (US)

Correspondence Address: Charles W. McHugh, 1010 Milby Rd., Arlington, TX, 76013, US

(21) Appl. No.: 10/763,069

(22) Filed: Jan. 20, 2004

Related U.S. Application Data
(60) Provisional application No. 60/441,390, filed on Jan. 20, 2003.

(51) Int. Cl. A61F 9/00

(52) U.S. Cl. 2/15

(57) ABSTRACT

An air deflector for upwardly moving ram air that—if allowed to continue—would impinge on the face of a motorcycle rider, said air rising from a region near the rider's feet as the motorcycle moves along a road. Air deflection is achieved by some kind of a structural body having sufficient density and rigidity to deflect ram air that is impinging on said body, in front of the rider's torso. There is also provided some kind of a structure for supporting the air-deflecting body in front of the rider's torso. The support structure should not constitute a safety hazard for the rider, and could be a frontal part of a leather vest that could otherwise be worn by the rider.

### CROSS-REFERENCE TO RELATED APPLICATION

This application contains information that was originally contained in Provisional Application Serial No. 60/ 441,390 filed Jan. 20, 2003.

### FIELD OF THE INVENTION

This invention relates generally to the riding of motorcycles, and to the deflection of ram air that would normally impinge on the face of a rider who is travelling at a significant speed. More specifically, it relates to a body-worn device, mounted about chest-high on the front of the rider; the main body of the device preferably has a stiffness similar to that of the visor of a baseball cap, and is sized as to deflect upwardly (over the rider) the ram air that normally moves upwardly from a low region near the rider's feet toward a rider's face.

### BACKGROUND OF THE INVENTION

It is well known that a motorcycle rider is subjected to ram air as the rider accelarates from a dead stop on a motorcycle—and exceeds a relatively modest speed, e.g., about 10 miles per hour (MPH). Engineers have attempted to deal with the obvious (frontal) ram air by affixing a transparent shield to the motorcycle's frame near the handlebars, so that ram air will be deflected horizontally, to the right and to the left, around the rider. However, it has been determined that there is a significant quantity of bothersome ram air that is not affected by such cycle-mounted windshields. That bothersome air is ram air that flows under the moving motorcycle and follows a general path upwardly from near the rider's feet, moving vertically along the front of the rider's body, and finally impinging on the rider's face. It is an object of this invention to deal with this vertically moving air stream, and to deflect it away from the rider's face—especially the rider's eyes.

Having recognized that a significant amount of bothersome air moves upwardly from a location near the biker's feet, it follows that it would be useful to have some kind of a deflector (similar in effectiveness to a windshield) in front of the biker's body—and between the biker's feet and his or her head. One way of mounting such a deflector might be to first install a pivotal bar to the frame of the motorcycle, similar to the pivotable security bars that hold passengers in their seats on the car of a roller-coaster. A desired air deflector could then be rigidly connected to the central portion of such a pivotal bar. However, such a hypothetical bar might restrain a biker to the extent that it would not be favored by most bikers. Too, a rigid bar or similar support could well constitute a safety hazard (to the rider) in the event of a traffic accident involving the bike. So a rigidly mounted air deflector across the chest of a biker is not considered to be practical.

If someone should suggest a body-mounted deflector for ram air, four other considerations should be addressed, namely, safety, appearance, weight and convenience. With regard to safety, a body-mounted air deflector should not introduce any hazard to motorcycle riding that does not already exist. For example, if a bike should accidentally strike an obstacle such as a curb head-on, an air deflector should not be constructed in such a way that it would cause chest injuries to a forwardly propelled rider. Ideally then, a deflector should be firm enough to deflect air but flexible enough to immediately yield in the event of an accident or the like. With regard to appearance, a desirable air deflector should not cause the wearer to take on the appearance of a “sissy,” especially if the biker is riding on what may be considered to be a “masculine” bike like a large HARLEY DAVIDSON™ bike. With regard to convenience, a body-worn deflector should not be unduly time consuming to put on or take off, and it should be easily worn by both men and women. Also, it should not uncomfortably heavy or interfere with routine and ordinary body movements of a biker, either when the biker is standing, sitting, walking or riding. The desired deflector should also be comfortable when worn in all kinds of weather and in all seasons. It is an object of this invention to satisfy these desirable characteristics of a proposed air deflector.

### BRIEF DESCRIPTION OF THE INVENTION

The invention comprises an air deflector for upwardly moving ram air that—if allowed to continue—would impinge on the face of a motorcycle rider, said air rising from a region near the rider's feet as the motorcycle moves along a road. As is commonly understood, the rider has feet and a torso that supports a head and a face, and the torso has a pair of forwardly oriented breasts.

Air deflection is achieved by some kind of a structural body having sufficient density and rigidity to deflect ram air that is impinging on said body. Preferably, the structural body has a length within the range of about 10 to 12 inches, so that it will extend across at least most of the front of the torso. When deployed to a working position the structural body also has a depth (from front to rear) within the range of about 3 to 4 inches.

There is also provided some kind of a structure for supporting the air-deflecting body in front of the torso of a motorcycle rider. The support is provided at a height that is approximately the same as the height of the rider's breasts, whereby ram air that is coming upwardly from a region near the rider's feet will be directed upwardly and forwardly—and away from the rider's face. The support structure should not constitute a safety hazard for the rider, and could be a frontal part of a leather vest that could otherwise be worn by the rider. On a hot day, when a leather garment might seem to be just a little bit too much, a foundation or support structure for an air deflector might be constructed out of light-weight jersey and, in appearance, might more nearly resemble a woman's sports bra.

### BRIEF DESCRIPTION OF THE SEVERAL FIGURES OF THE DRAWINGS

FIG. 1 is a side elevational view of a moving motorcycle and a rider in accordance with the prior art—with ram air impinging on the rider's face;

FIG. 2 is a side elevational view of an embodiment of the invention in which an air deflector has been deployed in front of the rider—to divert at least some of the ram air away from the rider's face;

FIG. 3 is a side elevation view of a biker standing astride a static bike, and showing an air deflector in its rest position—where it lies essentially “flat” against the front of the biker, so that it will not interfere with normal activities;

FIG. 4 is a side elevation view of a vest (without the wearer) in which upwardly moving ram air has caused an air deflector (i.e., a flap) to automatically rise in response to the presence of the ram air;

FIG. 5 is a side elevation view of a vest, similar to the view in FIG. 4, showing the position of the rotatable flap in the absence of ram air;

FIG. 6 is a front elevation view of an examplary vest with flap-type air deflectors in their rest position;

FIG. 7 is a diagrammatic view of the “anchor” part of a garment, said part being the part to which an air deflector is attached, and showing an alternate embodiment of an air deflector;

FIG. 8 is a cross-section view of the three-dimensional air deflector that is shown in FIG. 7;

FIG. 9 is a side elevation view of a generally planar air deflector that is created by placing a fabric over a stiffener (such as cardboard), and the air deflector being shown in its working position;

FIG. 10 is a side elevation view similar to FIG. 9, but showing the air deflector in its rest position—where it will remain until it is subjected to ram air and it automatically rotates upward to its working position;

FIG. 11 is a schematic view of one embodiment of the invention in which two flaps are identical in structure but have different indicia that are visible to persons standing in front of the wearer, so that a person may choose which flap he wants to use on a given day;

FIG. 12 is a front elevation view of a person standing errect and showing an alternate garment having similarites to a woman's sports bra, said garment being smaller than the vest shown in FIG. 6; and

FIG. 13 is a frontal view of a person with various locations on the body (e.g., eye level, waist, etc.) shown—to provide a definite frame of reference for where an air deflector of this invention is expected to be positioned.

### DESCRIPTION OF A PREFERRED EMBODIMENT OF THE INVENTION

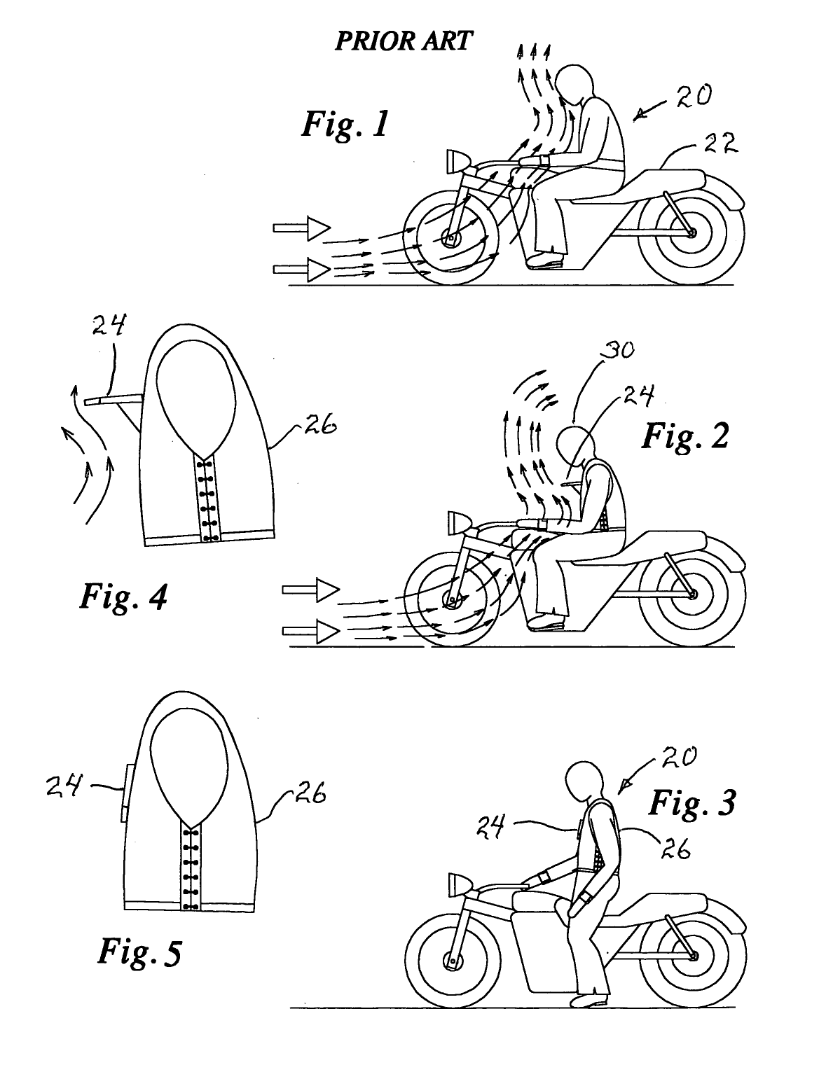

FIG. 1 is provided to show the general environment in which a motorcycle rider 20 typically enjoys the sport of bike riding—in accordance with prior art. Ram air is shown as if the rider and his bike 22 are moving to the left in the drawing, causing the ram air to rise from near the rider's feet and rolling up the rider's torso until it reaches his face. For simplicity in this figure, another quantity of ram air has been omitted, namely, the ram air that would be expected to have a horizontal orientation—where it would pass over the front of the bike and impinge directly on the front of the rider. It is this second quantity of ram air that a transparent windshield (when present on the bike) is designed to deflect, and this invention is not primarily concerned with such “straight line” ram air.

Referring next to FIG. 2, an air deflector 24 is shown deployed from the front of the rider 20 at an elevation between the rider's waist and the rider's head, i.e., approximately the same as that of the rider's breasts. The air deflector 24 has sufficient rigidity to cause the upcoming ram air to be deflected forwardly and upwardly over the rider's head 30. As a result of being deflected, this ram air does not impinge on the rider's face—and his eyes are effectively shielded.

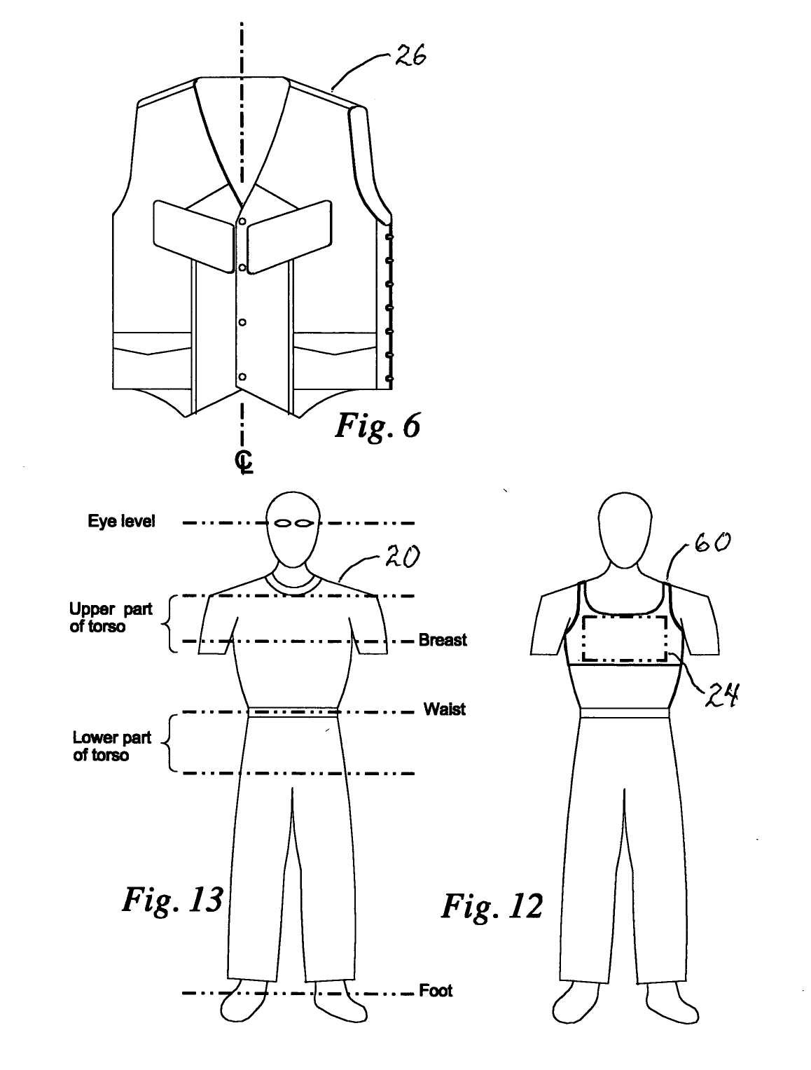

Referring next to FIGS. 3, 4, and 5, a preferred embodiment of the invention shows a vest 26 that is sized to fit comfortably on a rider 20. The air deflector 24 in this embodiment has two primary positions, one of which is a rest position in which it hangs downwardly where it will be generally parallel to the front of the vest. This position is shown in FIGS. 3 and 5. A frontal view of the vest is provided in FIG. 6. A preferred vest is made of relatively soft and comfortable material, and is shown at a size that will cause it to envelop most of the rider's torso. Leather is a common material for clothing and accessories for bikers, and leather could be advantageously used here, as could leather substitutes such as Naugahide™. The vest 26 has upper portions that drape over a rider's shoulders and support a major part of the vest over the rider's chest.

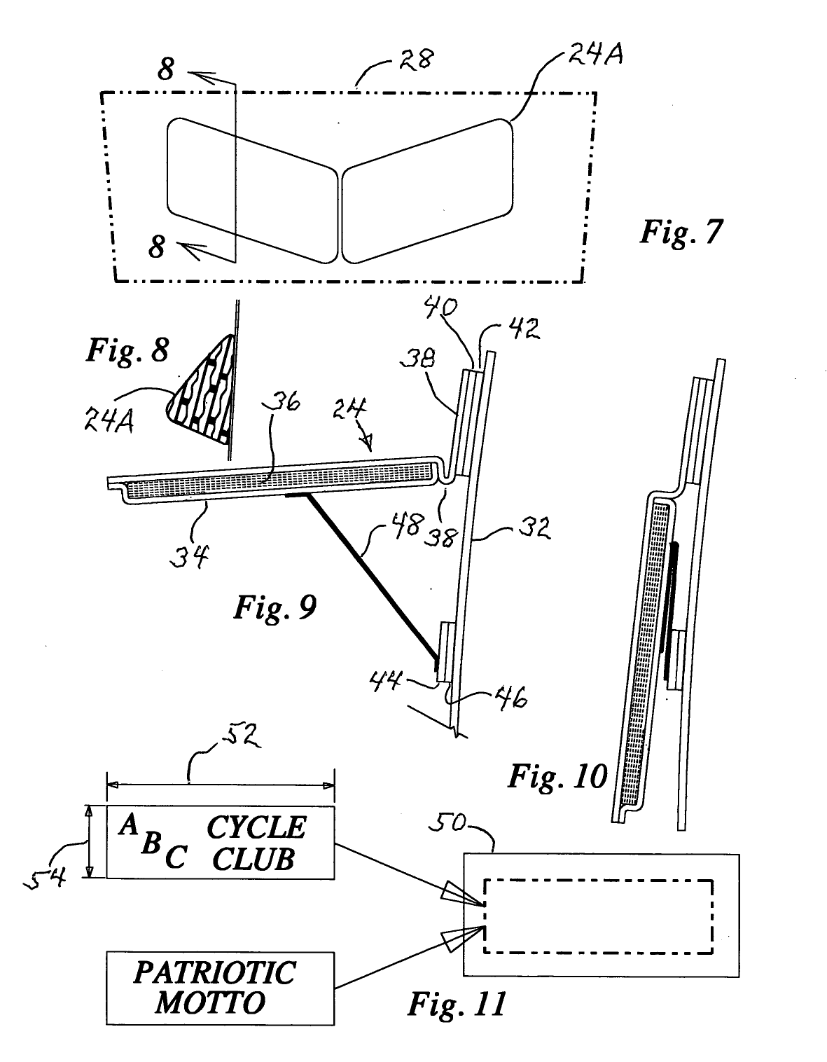

Referring next to FIG. 7 the aforementioned major part of the vest is identified by the rectangle 28. Without regard to its shape, this vest portion 28 may also be thought of as a non-rigid anchor for the deflector, in the sense that it holds the air deflector in place and prevents it from blowing away when ram air strikes it. Another feature of the embodiment shown in FIG. 7 is that the air deflector 24A is not planar (like the embodiment shown in FIG. 4). Instead, the three-dimensional deflector 24A is made of foam rubber or a natural sponge that has been shaped to extend far enough in front of a rider to function as an efficient deflector for ram air that is coming from below. (This structure is shown in FIG. 8) By virtue of its inherent softness, the deflector 24A with an air-impervious skin would not be expected to cause any injury to a rider in the event of an accident in which the rider is propelled forwardly.

Turning next to FIGS. 9 and 10, a generally planar air deflector 24 is shown larger than it is in FIG. 4, in order to better show how a flexible material might be used as the main material for a vest, as well as the exterior cover for a deflector. It will first be assumed that the material 32 (that is juxtaposed with the wearer's chest) is leather; a separate piece of the same material is then folded over to define an elongated sleeve 34. Into this sleeve 34 is inserted a corrugated cardboard core 36 that makes the combination rigid enough to serve as a deflector for ram air, but pliant enough to cause no injury to the wearer in the event of an accident. The upper part of the sleeve 34 has an elongated extension 38, and on the “inside” surface of this extension is affixed an elongated piece of the “hooks” 40 that constitute one half of a hook-and-loop fastener. The “loops” part 42 of the hook-and-loop fastener is permanently affixed to the outer surface of the vest, so that the air deflector might be selectively removable from the vest.

Another element that is connected to the vest with a hook-and-loop fastener 44, 46 is a flexible tether 48. The tether 48 has a length that will permit the air deflector 24 to rotate upwardly to a position in which it is essentially perpendicular to the rider's torso—when ram air pushes against the bottom of the air deflector. When the biker stops, as shown in FIG. 3, the air deflector 24 will rotate downwardly (about an effective hinge, along the length of the extension 38) and assume the position shown in FIG. 3.

Anther property of the invention is the capacity to provide indicia on the outer or exposed surface, for the purpose of showing the rider's association with some club, group or the like, etc. This property is shown in FIG. 11, where a rectangle 50 is again used to represent the “anchor” part of this combination, and two separate flaps are shown to the left. Either of the two flaps may be selectively attached to the anchor 50 with hook-and-loop fasteners, snaps or the like. If the vest wearer wakes up the next day and is in a different mood, a different flap may be chosen—and attached to the vest. Also shown in FIG. 11 are two dimensions which represent the two preferred dimensions for a one-piece air deflector. The first dimension (represented by the arrow 52) preferrably has a width of about ten inches; and a forward extension (represented by arrow 54) has a length of at least two inches. With these preferred dimensions there will be an effective area of at least twenty square inches of air-deflecting surface.

Turning next to FIG. 12, an alternate structure 60 to which a personal air deflector 24 may be attached is similar to a runner's singlet. It may be made of light-weight jersey, with perhaps a strap in the back to keep it from “riding up” on the wearer in response to wind loads on the garment. A similar garment might remind an observer of a woman's sports bra. And mothers who have ever fed solid food to a one-year old child might see the garment 60 and think of its similarity to a bib that hangs in front of the wearer as an “anchor” for the air deflector.

While it is believed that the named parts of a biker will be obvious to most readers, FIG. 13 has been provided to show various elevations of parts on a biker. The biker is shown in a standing position, but the relative positions will be essentially the same when the biker is sitting on a motorcycle.

While only a preferred embodiment of the invention—and some alternatives—have been shown herein in great detail, it should be understood that there are modifications that might be apparent to those skilled in the art. For example, flaps in the shape of rhomboids have been disclosed herein (in FIG. 6), but other shapes—when suitably mounted—could also be suitable. Hence, this invention should not be judged or limited by the few words in this specification; instead, it should be measured by the claims that are appended hereto.

What is claimed is:

1. In connection with a motorcycle rider who has a waist and a torso with a front, and the rider also having a head with eyes that are routinely subjected to ram air that arises from below the rider's torso when the motorcycle moves forwardly, the method of reducing discomfort that occurs when ram air impinges on the rider's eyes, comprising the steps of:
a) suspending an air deflector from the front of the rider's torso at an elevation between the rider's waist and the rider's head; and
b) causing the air deflector to project forwardly from the rider's torso so as to cause the ram air that strikes the deflector to be deflected instead of impacting the rider's eyes.

2. The method as claimed in claim 1 wherein the rider has breasts on the front of his torso, and the air deflector is suspended at an elevation that is approximately the same as the rider's breasts.

3. The method as claimed in claim 1 wherein the air deflector projects forwardly from the rider's torso for a distance of at least two inches, and the effective area of the air deflector against which ram air impinges being at least 20 square inches.

4. The method as claimed in claim 1 and further causing the air deflector to automatically project forwardly from the rider's torso in response to the presence of ram air, whereby the air deflector is automatically projected forwardly when it is needed and does not project forwardly when it is not needed.

5. The method as claimed in claim 1 wherein the air deflector is generally planar, and the air deflector is caused to project forwardly by rotating it about an effective hinge line that is immediately adjacent the front of the rider's torso.

6. The method as claimed in claim 1 and including the further step of affixing a tether line of fixed length to the air deflector, and causing the tether line to extend to its full length when the air deflector is projected forwardly so as to be essentially perpendicular to the rider's body.

7. The method of improving the comfort of a motorcycle rider by diverting at least a portion of the ram air that normally flows along the rider's body from near the rider's feet to the rider's face, comprising the steps of:
a) providing at least one flap to be worn at the front of the motorcycle rider, and said at least one flap having a rest position at which it hangs downwardly from the front of the rider's body, and said at least one flap having an operative position at which it protrudes forwardly from the front of the rider's body to function as an air deflector for ram air blowing upwardly along the front of the rider's body; and
b) restraining said at least one flap when it has been moved to its operative position by the presence of ram air blowing upwardly along the front of the rider's body, whereby at least a portion of the ram air is diverted over the head of the rider instead of impinging on the eyes of the rider.

8. The method as claimed in claim 7 and including the further step of dividing said at least one flap into two halves and placing each of the two halves on opposite sides of a centerline through the rider's body.

9. The method as claimed in claim 7 and including the further step of providing sufficient weight on said at least one flap so as to cause the flap to promptly fall to its rest position whenever ram air no longer contributes a supporting force to said at least one flap.

10. The method as claimed in claim 7 and including the further step of attaching the air deflector to the front of a garment that is adapted to be worn by a motorcycle rider, whereby the air deflector may be removed by the step of removing the garment.

11. A device to be worn by a motorcycle rider, the rider having a head and a torso with upper and lower parts, and the torso also having a front and a back, and there being ram air that rushes upwardly along the front of the rider when the motorcycle is moving forwardly, comprising:
a) an anchor part adapted to be supported in a static manner over the front of the upper part of a rider's torso;
b) at least one air-impervious flap having a rectilinear shape with a linear edge that is secured to the anchor part in such a way that the flap can assume either one of two positions, the first position being one in which the flap hangs downwardly so as to be generally parallel to the anchor part, and the second position being one in which the flap extends forwardly from the anchor part and is generally perpendicular to the anchor part, whereby ram air will be at least partially deflected over the head of the rider when the flap is in its second position; and
c) a restraining member for holding the flap in its second position when the motorcycle is moving forward and ram air is acting on the flap so as to rotate it upwardly.

12. The device as claimed in claim 11 wherein the restraining member is a flexible member with first and second ends, and the first end being secured to the anchor member, and the second end being secured to the flap.

13. The device as claimed in claim 11 wherein the anchor part constitutes a frontal part of a garment that can be worn by the motorcycle rider.

14. The device as claimed in claim 11 wherein the anchor part constitutes part of a sleeve-less vest that is configured to be worn by a rider over other clothing.

15. The device as claimed in claim 11 wherein the anchor part and the flap are made of the same material.

16. The device as claimed in claim 11 wherein both the anchor part and the flap are made of leather.

17. The device as claimed in claim 11 where there are two air-impervious flaps, each of which has the shape of a rhomboid.

18. The device as claimed in claim 11 wherein there are two air-impervious flaps, each of which is generally planar, and the combined area of the two flaps being at least 20 square inches.

19. The device as claimed in claim 11 wherein said at least one flap is relatively flexible, whereby pushing it against the torso of the motorcycle rider will not cause any injury to the rider, and whereby a vehicular accident involving the rider's motorcycle will not contribute to an injury to the rider.

20. The device as claimed in claim 11 wherein the flap is selectively removeable and replaceable with a similarly configured flap.

21. The device as claimed in claim I wherein the flap has an outer surface that is visible to persons other than the motorcycle rider when the flap is in its first position, and further including advertising indicia on said outer surface that is visible to persons other than the motorcycle rider.
